(19) United States
(12) Patent Application Publication
(10) Pub. No.: US 2023/0181810 A1
(43) Pub. Date: Jun. 15, 2023

(54) SURGICAL DEVICE ACTIVATION DETECTION USING CURRENT SENSING

(71) Applicant: Medos International Sarl, Le Locle (CH)

(72) Inventors: Oliver Dumas, East Sandwich, MA (US); Michael Confoey, Westwood, MA (US); Pedro John Miguel, Fall River, MA (US)

(21) Appl. No.: 17/550,586

(22) Filed: Dec. 14, 2021

(51) Int. Cl. A61M 1/00; A61B 18/14; A61B 17/16

(52) U.S. Cl. CPC A61M 1/74; A61B 18/14; A61B 17/1659; A61B 2018/00577

(57) ABSTRACT

In general, devices, systems, and methods for surgical device activation detection using current sensing are provided. In an exemplary embodiment, a surgical system includes a surgical pump, a detector, and a surgical device. The detector is configured to sense changes in electrical power in an AC power line from an AC power source to the surgical device to detect activation of the surgical device. In response to the detection activation, the pump can properly provide suction pressure to the surgical device to allow the surgical device to provide aspiration at a surgical site during performance of a surgical procedure.

### FIELD

The present disclosure generally relates to surgical device activation detection using current sensing.

### BACKGROUND

Arthroscopic pumps are used in a variety of surgical procedures in connection with a variety of functions, such as soft tissue ablation, contouring, cutting, coagulation, and temperature control. During performance of a surgical procedure an arthroscopic pump may provide irrigation (inflow) of fluid such as saline through inflow tubing to a surgical site, e.g., a joint of a patient, and aspiration (outflow) of fluid through outflow tubing from the surgical site. Such pumps can control fluid pressure at the joint to help provide joint distension for easy access, maintain good visibility, and/or to control bleeding.

Some pumps allow an aspiration device to be connected to the pump to facilitate outflow of fluid from the surgical site. Such pumps include multiple routes of outflow. First outflow tubing allows aspiration of pressurized fluid therethrough from the surgical site when the aspiration device is not connected to the pump. Second outflow tubing allows aspiration of pressurized fluid therethrough from the surgical site when the aspiration device is connected to the pump and is active, such as by a trigger of the device being actuated to activate aspiration operation of the device. Different aspiration devices may be connected to the pump. However, traditionally, the pump cannot detect operation of every possible aspiration device that may be connected to the pump, e.g., because not all aspiration devices have the same manufacturer as the pump. The pump may therefore not properly function with the aspiration device.

Accordingly, there remains a need for improved devices, systems, and methods for arthroscopic pumps.

### SUMMARY

In general, devices, systems, and methods for surgical device activation detection using current sensing are provided.

In one aspect, a surgical system is provided that in one embodiment includes a detector configured to be operatively connected to each of an AC power source, a surgical pump, and a surgical device. The surgical device is configured to be releasably coupled to the surgical pump. The detector includes a housing external to the pump and to the surgical device. The detector also includes a sensor and a circuit housed in the housing. The sensor is configured to sense electrical power received by the detector from the AC power source. The circuit is configured to determine if the sensed electrical power exceeds a threshold and, if the sensed electrical power is determined to exceed the threshold, to transmit a signal to the surgical pump that causes the surgical pump to provide suction pressure to the surgical device thereby allowing the surgical device to suction fluid from a surgical site.

The surgical system can have any number of variations. For example, the sensed electrical power exceeding the threshold can be indicative of the surgical device being actuated to suction fluid.

For another example, the sensed electrical power exceeding the threshold can be indicative of the surgical device being in an active state in which the surgical device is operational to provide aspiration, and the sensed electrical power not exceeding the threshold can be indicative of the surgical device being in an inactive state in which the surgical device is not operational to provide aspiration.

For yet another example, the threshold can be pre-programmed and stored at the detector.

For still another example, at least one of the detector and the surgical pump can include an adjuster configured to allow a user to set the threshold.

For another example, the circuit can be configured to dynamically set the threshold based on the sensed electrical power.

For yet another example, the sensor can include a current sensor, and the sensed electrical power can include current.

For another example, the sensed electrical power can include a magnetic field.

For still another example, the sensor can be configured to repeatedly sense electrical power received over time by the detector from the AC power source, and, if the sensed electrical power is determined to not exceed the threshold, the circuit can be configured to determine if a next sensed electrical power exceeds the threshold.

For yet another example, the surgical pump can include an arthroscopic pump, and the surgical site can be at a joint of a patient.

In another embodiment, a surgical system includes a surgical device configured to be operatively connected to surgical source equipment configured to provide operational functionality to the surgical device operatively connected thereto. The operational functionality allows the surgical device to perform a function at a surgical site. The surgical system also includes a housing that is external to the surgical device and to the surgical source equipment and that is configured to be operatively coupled to the surgical device, to the surgical source equipment, and to an AC power source. The surgical system also includes a sensor housed in the housing and configured to sense electrical power received at the housing from the AC power source. The surgical system also includes a circuit housed in the housing and configured to determine if the sensed electrical power exceeds a threshold and, if the sensed electrical power is determined to exceed the threshold, to transmit a signal to the surgical source equipment that causes the surgical source equipment to provide the operational functionality to the surgical device and thereby allowing the surgical device to perform the function at the surgical site.

The surgical system can have any number of variations. For example, the sensed electrical power exceeding the threshold can be indicative of the surgical device being in an active state in which the surgical device is operational to perform the function, and the sensed electrical power not exceeding the threshold can be indicative of the surgical device being in an inactive state in which the surgical device is not operational to perform the function.

For another example, the threshold can be pre-programmed.

For yet another example, the surgical system can also include an adjuster configured to allow a user to set the threshold.

For still another example, the circuit can be configured to dynamically set the threshold based on the sensed electrical power.

For another example, the current sensor can be configured to repeatedly sense electrical power received over time at the housing from the AC power source, and, if the sensed electrical power is determined to not exceed the threshold, the circuit can be configured to determine if a next sensed electrical power exceeds the threshold.

For yet another example, the sensor can include a current sensor, and the sensed electrical power can include current.

For another example, the sensed electrical power can include a magnetic field.

For yet another example, the surgical source equipment can include a surgical pump, the operational functionality can include a suction force, and the function can include suctioning fluid from the surgical site. In some embodiments, the surgical pump can include an arthroscopic pump, and the surgical site can be at a joint of a patient.

For still another example, the surgical device can include an electrode, the surgical source equipment can include a generator, the operational functionality can include RF energy, and the function can include delivering RF energy to tissue at the surgical site via the electrode.

In another aspect, a surgical method is provided that in one embodiment includes sensing, using a sensor housed in a housing of a detector, electrical power received at the detector from an AC power source. The detector is operatively coupled to a surgical pump and is external to the surgical pump. The surgical method also includes determining, using a circuit housed in the housing of the detector, if the sensed electrical power exceeds a threshold. The surgical method also includes, if the sensed electrical power is determined to exceed the threshold, transmitting a signal from the detector to the surgical pump that causes the surgical pump to provide a suction force to a surgical device releasably coupled to the surgical pump such that the surgical device suctions fluid from a surgical site.

The surgical method can vary in any number of ways. For example, the sensed electrical power exceeding the threshold can be indicative of the surgical device being actuated to suction fluid.

For another example, the sensed electrical power exceeding the threshold can be indicative of the surgical device being in an active state in which the surgical device is operational to provide aspiration, and the sensed electrical power not exceeding the threshold can be indicative of the surgical device being in an inactive state in which the surgical device is not operational to provide aspiration.

For yet another example, the threshold can be pre-programmed and stored at the detector.

For still another example, the threshold can be set by a user via an adjuster of the detector.

For another example, the threshold can be set by a user via an adjuster of the surgical pump.

For yet another example, the threshold can be dynamically set by the circuit based on the sensed electrical power.

For another example, the surgical method can also include repeatedly sensing, using the sensor, electrical power received over time by the detector from the AC power source, and, if the sensed electrical power is determined to not exceed the threshold, determining, using the circuit, if a next sensed electrical power exceeds the current threshold.

For yet another example, the sensor can include a current sensor, and the sensed electrical power can include current.

For another example, the sensed electrical power can include a magnetic field.

For still another example, the surgical pump can include an arthroscopic pump, and the surgical site can be at a joint of a patient.

### BRIEF DESCRIPTION OF DRAWINGS

This disclosure will be more fully understood from the following detailed description taken in conjunction with the accompanying drawings, in which:

FIG. 1 is a block diagram of one embodiment of a pump system;

FIG. 2 is a perspective view of a portion of the pump system of FIG. 1;

FIG. 3 is a schematic view of one embodiment of a detector of the pump system of FIG. 1;

FIG. 4 is a flowchart showing one embodiment of a method of using the pump system of FIG. 1; and

FIG. 5 is a schematic view of one embodiment of a detector of a radiofrequency system.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

In general, devices, systems, and methods for surgical device activation detection using current sensing are provided. In an exemplary embodiment, a surgical system includes a surgical pump, a detector, and a surgical device. The detector is configured to sense changes in electrical power in an AC power line from an AC power source to the surgical device to detect activation of the surgical device during performance of a surgical procedure. In response to the detection activation, the pump can properly provide suction pressure to the surgical device to allow the surgical device to provide aspiration at a surgical site. Electrical power consumption by the surgical device is higher when the surgical device is operational, e.g., is active and providing suction, compared to when the surgical device is not operational, e.g., is inactive and not providing suction. Thus, the detector being configured to sense electrical power changes in the AC power line allows for detection of when the surgical device is operational (higher current) or not operational (lower current).

In an exemplary embodiment, the electrical power is current. Voltage remains constant, but current will vary. In another exemplary embodiment, the electrical power is magnetic field. Magnetic field is proportional to current, which will vary such that the magnetic field will correspondingly vary.

The surgical device is configured to be releasably coupled to the surgical pump such that a second surgical device can subsequently be releasably coupled to the surgical device. Different surgical devices can be connected to the surgical pump, such as surgical devices from different manufacturers than the surgical pump. However, the surgical pump can be configured to detect activation of only certain surgical device(s), such as those surgical device(s) having a same manufacturer as the surgical pump and thus being designed for communication with the pump. The pump may therefore not be able to perform properly with some surgical devices even though those surgical devices can be operatively connected to the surgical pump and therefore appear as if they should perform properly with the pump. For example, the surgical pump may not be able to determine that the surgical device has been actuated for suction, so the surgical pump does not begin providing suction force to the surgical device despite user request for suction functionality. The surgical device activation detection using electrical power sensing described herein may allow all these different surgical devices to be used with the pump, even those surgical devices whose activation the pump is not configured to detect.

Surgeons often have preferred surgical devices and preferred surgical pumps, each of which may be from a different manufacturer or otherwise not be compatible for proper communication of surgical device operation. The surgical device activation detection using electrical power sensing described herein may allow for surgeons to use their preferred surgical pump with their preferred surgical device even if the surgical pump itself cannot detect operation of the surgical device.

Some surgeons may only have access to certain surgical devices and certain surgical pumps, each of which may be from a different manufacturer or otherwise not be compatible for proper communication of surgical device operation. The surgical device activation detection using electrical power sensing described herein may allow for surgeons to use the surgical pump on hand with the surgical device on hand even if the surgical pump itself cannot detect operation of the surgical device.

In an exemplary embodiment, the detector is external to the surgical pump. The detector can therefore be used with existing surgical pumps without the pump needing to be modified.

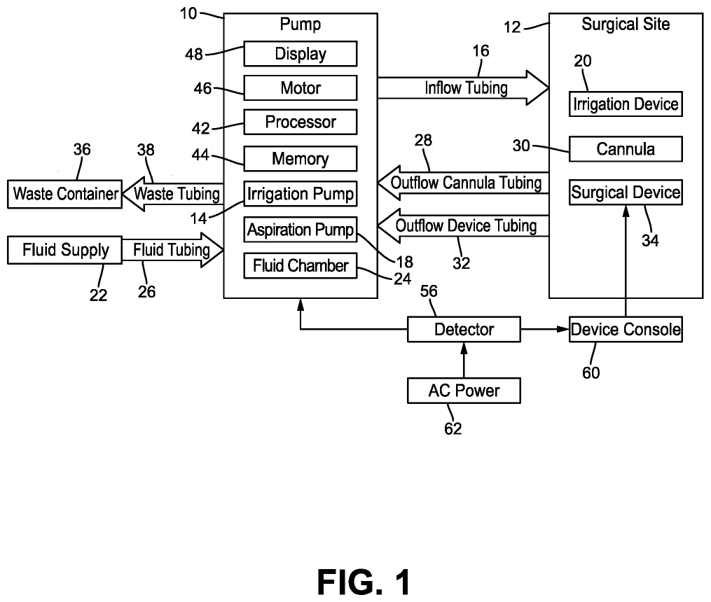

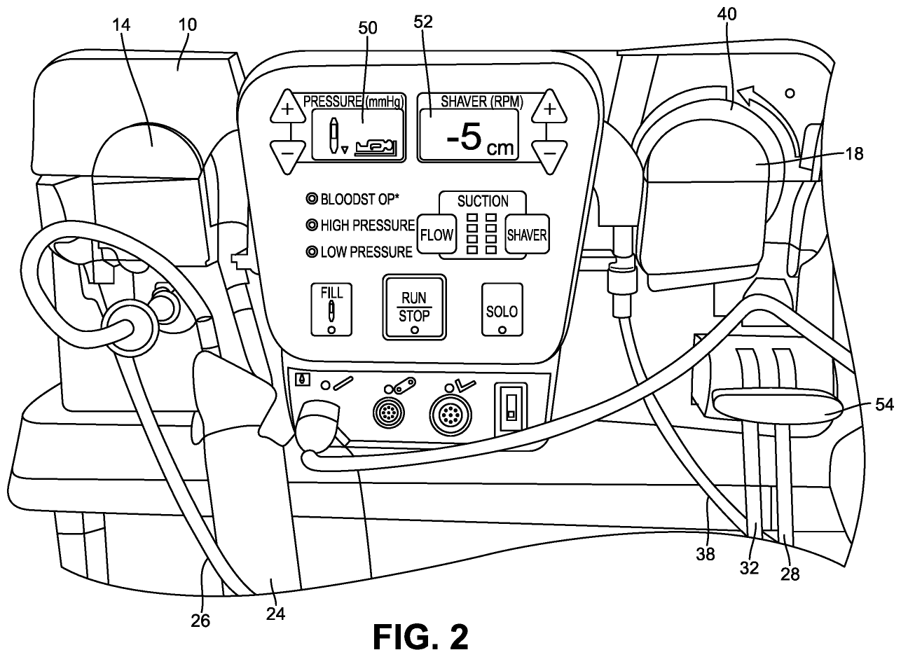

The devices, systems, and methods described herein can be used with a variety of surgical pumps. One example of a surgical pump is the FMS VUE® pump of the FMS VUE® Fluid Management and Tissue Debridement System available from DePuy Mitek of Raynham, Mass. Other examples of surgical pumps are described in U.S. Pat. Pub. No. 2017/0120039 entitled “Anti-Clogging Fluid Management System” published May 4, 2017 and U.S. Pat. No. 10,874,766 entitled “Methods, Systems, And Devices For Joint To Pump Elevation Level User Interfaces, Autocalibration For Joint Elevation, And Joint Pressure Estimation” issued Dec. 29, 2020, which are hereby incorporated by reference in their entireties. Another example of a surgical pump is illustrated in FIGS. 1 and 2.

FIGS. 1 and 2 illustrate one embodiment of a pump system including an arthroscopic pump 10 configured to pump fluid to and from a surgical site 12. In an exemplary embodiment the surgical site 12 is at a joint such as the knee or shoulder. The pump 10 can have a variety of configurations. In the illustrated embodiment the pump 10 includes an irrigation pump 14 configured to pump fluid to the surgical site 12 through inflow tubing 16 and includes an aspiration pump (suction pump) 18 configured to pump fluid from the surgical site 12 through outflow tubing. Each of the inflow tubing 16 and the outflow tubing can have an associated sheath.

The inflow tubing 16 is configured to be operatively connected to an irrigation device 20 configured to be used at the surgical site 12 such that fluid can be provided to the surgical site 12 through the irrigation device 20. The irrigation device 20 can have any of a variety of configurations, as will be appreciated by a person skilled in the art. The inflow tubing 16 is configured to be operatively connected to a fluid supply 22, such as a bag or other container of saline or other fluid, external to the pump 10 via a fluid chamber (fill chamber or reservoir) 24 of the pump 10 and fluid tubing 26 extending between the fluid supply 22 and the pump 10. The fluid chamber 24 may help smooth fluid flow into the inflow tubing 16 and/or may allow for pressure sensing at a top of the fluid chamber 24 to facilitate determining of fluid pressure at the surgical site 12. Fluid pressure can be determined in any number of ways and, in at least some embodiments, can be determined at the pump 10. For example, various embodiments of determining fluid pressure are described in previously mentioned U.S. Pat. No. 10,874,766 entitled “Methods, Systems, And Devices For Joint To Pump Elevation Level User Interfaces, Autocalibration For Joint Elevation, And Joint Pressure Estimation” issued Dec. 29, 2020.

The outflow tubing includes outflow cannula tubing 28 configured to be operatively connected to a cannula 30 configured to be used at the surgical site 12 such that fluid at the surgical site 12 can be suctioned from the surgical site 12 into the cannula 30 and then into the outflow cannula tubing 28. The cannula 30 can have any of a variety of configurations, as will be appreciated by a person skilled in the art. The outflow tubing also includes outflow device tubing 32 configured to be operatively coupled to a surgical device 34 configured to be used at the surgical site 12 such that fluid at the surgical site 12 can be suctioned from the surgical site 12 into the surgical device 34 and then into the outflow cannula tubing 28. The surgical device 34 is configured to be releasably coupled to the pump 10. The surgical device 34 can have any of a variety of configurations, as will be appreciated by a person skilled in the art. The surgical device can be a dedicated suction device (aspiration device) or can be a device with suction functionality. In this illustrated embodiment, the surgical device 34 is a shaver. Various embodiments of shavers are further described, for example, in U.S. Pat. No. 9,782,193 entitled “Tissue Shaving Device Having A Fluid Removal Path” issued Oct. 10, 2017 and U.S. Pat. No. 9,186,166 entitled “Tissue Shavers” issued Nov. 17, 2015, which are hereby incorporated by reference in their entireties.

The outflow cannula tubing 28 and the outflow device tubing 32 are each configured to be operatively coupled to a waste container 36 external to the pump 10 that is configured to collect therein fluid (and any solid matter) suctioned from the surgical site 12 through the outflow cannula tubing 28 and the outflow device tubing 32. As in this illustrated embodiment, the outflow cannula tubing 28 and the outflow device tubing 32 can merge into a single outflow tube 40 at the pump 10, which allows waste tubing 38 that is configured to operatively connect the waste container 36 to the outflow tubing to be a single tube. One of the outflow cannula tubing 28 and the outflow device tubing 32 can be open or active at a time with the other of the outflow cannula tubing 28 and the outflow device tubing 32 pinched off or closed, e.g., with a pinch valve 54, such that fluid is being suctioned toward the waste container 36 through only one of the outflow cannula tubing 28 and the outflow device tubing 32 at a time.

The pump 10 also includes a processor 42 that is configured to control the irrigation pump 14 and the aspiration pump 18, a memory 44 configured to store instructions therein that are executable by the processor 42, and a motor 46 configured to drive the irrigation pump 14 and the aspiration pump 18. The processor 42 is configured to control operation of the motor 46, e.g., by executing instructions stored in the memory 44. The processor 42 and the memory 44 are shown as part of the pump 10 in FIG. 2, but in other embodiments the processor 42 and/or the memory 44 can be located elsewhere in the pump system.

The pump system also includes a user interface configured to facilitate user interaction with the pump 10. The user interface includes a display 48 that includes a first display 50 configured to display joint pressure (in mmHg in this illustrated embodiment) in real time with use of the pump 10 during performance of a surgical procedure. The fluid pressure shown on the first display 50 is the pressure of fluid at the surgical site 12, e.g., pressure as determined by the pump 10. The display 48 also includes a second display 52 configured to display surgical device speed (shaver speed in revolutions per minute (RPM) in this illustrated embodiment).

The user interface also includes user controls configured to receive input from the user to control various pump functions. In the illustrated embodiment the user controls include depressible +/− (up/down) arrow buttons to control adjustment of information on the first display 50, and depressible +/− (up/down) arrow buttons to control adjustment of information on the second display 52. In other embodiments controls other than buttons may be used for the display 48, such as knobs, dials, levers, a keypad, or the like. Aspiration pump flow rates are controlled by buttons labelled FLOW and SHAVER. The FLOW flow button is configured to be activated with a foot pedal, and the SHAVER flow button is configured to be activated when a shaver, e.g., the surgical device 34, is enabled. The illustrated embodiment also includes a key that enables filling of the fluid chamber 24 and a key that enables SOLO (irrigation pump 14 only) mode. In other embodiments one or both of the FLOW and SHAVER buttons can have another form, such knobs, dials, levers, or the like. The illustrated user interface also includes lights, e.g., light-emitting diodes (LEDs) or the like, that illuminate to indicate various conditions such as bloodstop, high pressure warning, low pressure alarm, FLOW flow rate, SHAVER flow rate, chamber fill enabled, and pump on/off (run/stop).

As shown in FIG. 1, the pump system includes a detector 56. The detector 56 is configured to be operatively connected to the pump 10, to the surgical device 34 via a device console 60, and to AC power 62, e.g., an AC power source such as a wall outlet or other source. The AC power 62 is configured to provide power to the device console 60, and thus to the surgical device 34, via the detector 56. Although the device console 60 is shown in FIG. 1 as being located outside of the surgical site 12, the device console 60 can be integrally formed with the surgical device 34 and thus be located at the surgical site 12 with the surgical device 34.

The detector 56 is configured to, based on AC current level, detect actuation of the surgical device 34 to provide suction. As discussed herein, current level can be indicated with a sensed current or a sensed magnetic field. Detecting actuation of the surgical device 34 allows for multiple events to occur, as discussed further below. The surgical device 34 can be actuated in a variety of ways, depending on the particular surgical device 34. For example, the surgical device 34 can be actuated by pulling a trigger of the surgical device 34, pushing a button on the surgical device 34, moving a lever on the surgical device 34, or by another action. In an exemplary embodiment the surgical device 34 is actuated manually. Manual activation of the surgical device 34 can thus be configured to cause the detector 56 to automatically electronically detect that the surgical device 34 has been actuated and cause a plurality of events to occur.

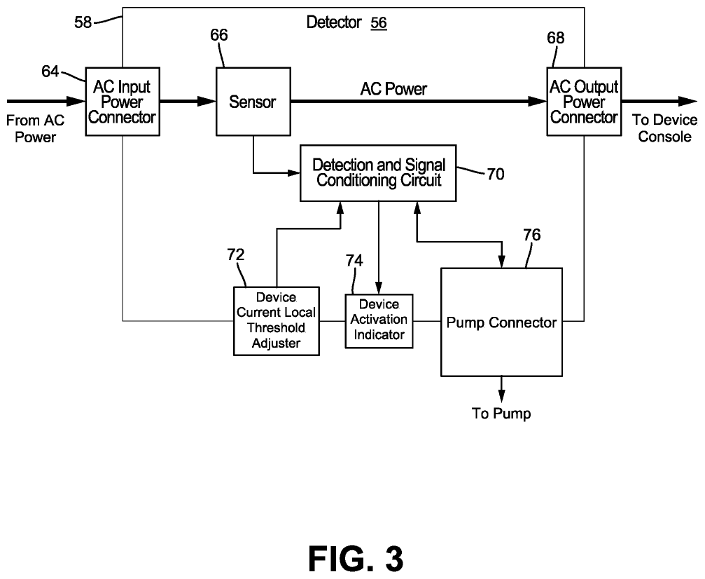

The detector 56 is also shown in FIG. 3 and includes a housing 58, such as a metal and/or plastic box or casing, configured to house various components of the detector 56. The detector 56 includes an AC input power connector 64 configured to operatively connect to the AC power 62. In an exemplary embodiment, the AC input power connector 64 includes a wired connector such that the detector 56 is operatively connected to the AC power 62 via a wired connection, e.g., by a cable extending from the detector 56 being plugged into or otherwise attached to the AC power 62.

The detector 56 also includes a sensor 66 configured to sense electrical power indicative of the AC current received at the detector 56 from the AC power 62 via the AC input power connector 64. The sensed electrical power can be, for example, sensed current or a sensed magnetic field. The sensor 66 can include, for example, a Hall effect sensor. For another example, the sensor 66 can include an inductive sensor.

The detector 56 also includes an AC output power connector 68 configured to operatively connect to the device console 60. The AC output power connector 68 allows AC power received at the detector 56 from the AC power 62 (via the AC input power connector 64) to be output to the surgical device 34 (via the device console 60) to power the surgical device 34. In an exemplary embodiment, the AC output power connector 68 is operatively connected to the device console 60 via a wired connection, such as by a cable extending from the device console 60 being plugged into or otherwise attached to the AC output power connector 68 of the detector 56.

The detector 56 also includes a detection and signal conditioning circuit 70. The circuit 70 is configured to receive a signal from the sensor 66 indicating the electrical power sensed by the sensor 66. The circuit 70 is also configured to condition the signal. Additionally, as discussed further below, the circuit 70 is configured to compare the sensed electrical power with a threshold electrical power to facilitate control of the surgical device 34. In an exemplary embodiment, the threshold electrical power is a threshold current since the sensed electrical power can be indicative of current either by sensing current or sensing a magnetic field that is indicative of current.

The detector 56 also includes a device current local threshold adjuster 72 configured to be in communication with the circuit 70. The device current local threshold adjuster 72 is configured to be manually adjusted by a user to adjust the threshold current used by the circuit 70 and stored at the detector 56, e.g., in a memory thereof. The device current local threshold adjuster 72 can have a variety of configurations. For example, the device current local threshold adjuster 72 can include a knob configured to be rotated by a user to manually adjust a value of the threshold current. For another example, the device current local threshold adjuster 72 can include depressible +/− (up/down) arrow buttons to allow a user to manually adjust a value of the threshold current. For yet another example, the device current local threshold adjuster 72 can include a keypad configured to allow a user to input a numeric value of the threshold current.

The detector 56 also includes a device actuation indicator 74 configured to be in communication with the circuit 70. The device actuation indicator 74 is configured to indicate to a user that the surgical device 34 is powered on and receiving AC power such that the surgical device 34 is in operation to suction fluid. The indicator 74 can include a visual indicator such as a light (light emitting diode or other type of light) configured to illuminate to indicate that the surgical device 34 is powered on. The indicator 74 can additionally or alternatively include an audible indicator such as a speaker configured to emit a beep, a series of beeps, a musical sequence, or other sound to indicate that the surgical device 34 is powered on.

The detector 56 also includes a pump connector 76 configured to be in communication with the circuit 70 and to operatively connect to the pump 10. The pump connector 76, when operatively connected to the pump, allows the detector 56 to transmit signals to the pump 10 and to receive signals from the pump 10, as discussed further below. The pump connector 76 can be operatively connected to the pump 10 via a wired or a wireless connection. In the case of a wired connection, for example, a cable extending from the pump 10 can be plugged into or otherwise attached to the pump connector 76. In the case of a wireless connection, any of a variety of wireless communication protocols can be used.

As mentioned above, the current threshold can be set by a user via the detector's device current local threshold adjuster 72. Such current threshold adjustment occurs local to the detector 56. In some embodiments, the detector 56 omits the device current local threshold adjuster 72. In such embodiments, the current threshold adjustment can occur remote from the detector 56 by being performed at the pump 10. The remote current threshold adjustment is performed similar to the local current threshold adjustment except is performed at the pump 10. The pump 10 can therefore include a device current remote threshold adjuster that is similar to the device current local threshold adjuster 72 discussed above, e.g., include a knob, depressible +/− (up/down) arrow buttons, a keypad, or other adjuster. In such embodiments, the pump 10 is configured to communicate the user-set current threshold to the detector 56 via the pump connector 76.

In some embodiments, the pump system includes the current local threshold adjuster 72 at the detector 56 and the current remote threshold adjuster at the pump 10. A user therefore has flexibility in deciding whether to adjust the current threshold via the detector 56 or via the pump 10. Some users may be more familiar with one of the detector 56 and the pump 10 than the other.

In some embodiments, the current threshold can be pre-programmed at the detector 56, e.g., pre-stored in a memory thereof, as a default current threshold. Having a default current threshold may ensure that a current threshold is stored at the detector 56 so as to not be reliant on a user remembering to set the current threshold. The default current threshold can be, for example, in a range of about 0.1 to about 2 amps, as a shaver typically takes about 30 volts.

In some embodiments, the default current threshold is not adjustable. In such embodiments, the pump system does not include either of the current local threshold adjuster 72 at the detector 56 and the current remote threshold adjuster at the pump 10. Having a non-adjustable default current threshold may allow for a simpler, less expensive pump system. In other embodiments, the default current threshold is adjustable. In such embodiments, the pump system includes one of or both of the current local threshold adjuster 72 at the detector 56 and the current remote threshold adjuster at the pump 10. The user's local or remote adjustment of the current threshold overrides the default current threshold. A user therefore may, but need not, adjust the adjustable default current threshold because if the user does not perform the adjustment, the detector 56 will use the default current threshold. Having an adjustable default current threshold may allow a user to make the detector 56 more sensitive (lower current threshold) or less sensitive (higher current threshold) to detecting activation of the surgical device 34.

In some embodiments, the detector 56 is configured to automatically set the current threshold. In such embodiments, the detector 56, e.g., the circuit 70 thereof, can use an active current corresponding to the surgical device 34 being in an active or actuated state in which the surgical device 34 is operational, e.g., is providing suction, and an inactive current corresponding to an inactive or standby state in which the surgical device 34 is not operational, e.g., is not providing suction. The sensor 66 is configured to monitor the active current and the inactive current, as discussed further below. The detector 56 can calculate the current threshold by, for example, subtracting the inactive current from the active current and halving the result as shown below:

$$\frac{active current - inactive current}{2} = current threshold$$

The detector 56 being configured to automatically set the current threshold allows the current threshold to be set based on the particular AC power 62 and the particular surgical device 34, which may eliminate the possibility of the detector 56 making an erroneous determination of the surgical device's state. Until the detector 56 has enough data to calculate the current threshold, e.g., has data indicating the active current and data indicating the inactive current, the detector 56 can use the default current threshold, which as discussed above can be a pre-programmed value or a value set by a user.

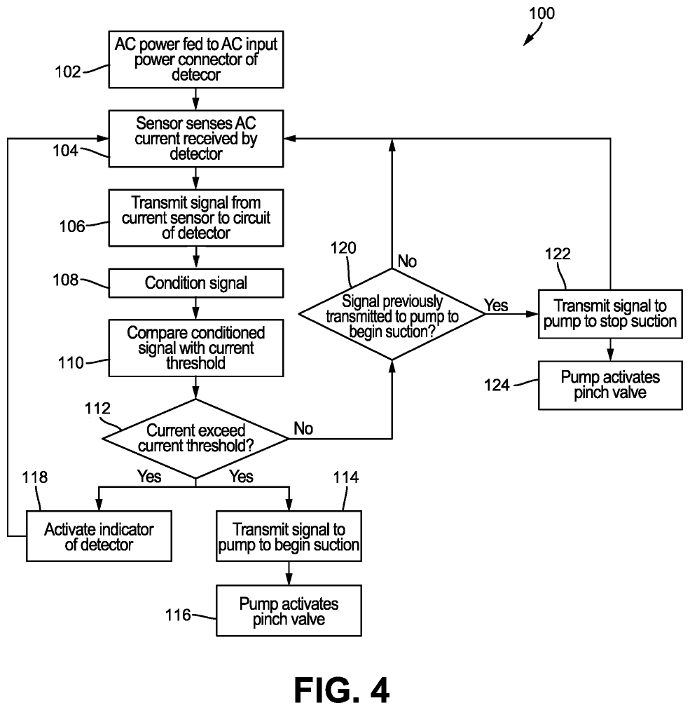

FIG. 4 illustrates one embodiment of a method 100 of using the detector 56 during performance of a surgical procedure. In general, the method 100 senses current changes in the AC power line from the AC power 62 to the device console 60 to detect activation of the surgical device 34 and to control the pump 10 accordingly. In the method 100 the detector 56 is operatively connected to the AC power 62 (e.g., via the AC input power connector 64), to the device console 60 (e.g., via the AC output power connector 68), and to the pump 10 (e.g., via the pump connector 76). Although the method 100 is described with respect to the detector 56 of FIG. 3 and the pump system of FIGS. 1 and 2, the method 100 can be similarly implemented using other pump systems and detectors as described herein.

In the method 100, the AC power 62 is fed 102 to the AC input power connector 64 of the detector 56. The AC power 62 feed 102 can begin either when the detector 56 is powered on or when the pump 10, which is operatively connected to the detector 56 is turned on.

The sensor 66 of the detector 56 senses 104 the AC power received at the detector 56 via the AC input power connector 64. The sensor 66 transmits 106 a signal indicative of the sensed power to the circuit 70 of the detector 56. The circuit 70 conditions 108 the received signal. The signal conditioning 108 generates a DC signal (e.g., a DC linear voltage) representative of the sensed AC current, which as discussed herein can be sensed directly (e.g., using a current sensor) or indirectly (e.g., by sensing magnetic field).

The circuit 70 compares 110 the conditional signal with the current threshold. As mentioned above, the current threshold can be set in any of a variety of ways. If the comparison 110 determines 112 that the sensed current exceeds the current threshold, the surgical device 34 is presumed to be in the active or actuated state in which the surgical device 34 is operational, e.g., is providing suction. Current consumption by the surgical device 34 increases when the surgical device 34 is in the active or actuated state, as compared to when the surgical device 34 is an inactive or standby state in which the surgical device 34 is not operational, e.g., is not providing suction. The current exceeding the current threshold thus indicates that the surgical device 34 is receiving high enough power to be operational and therefore that the surgical device 34 has been actuated.

Also in response to determining 112 that the current exceeds the current threshold, the detector 56 transmits 114 an instruction signal to the pump 10 via the pump connector 76. In response to receiving the instruction signal, the pump 10 activates 116 the pinch valve 54 so that suction is provided through the outflow device tubing 32. If the motor 46 is not already driving the aspiration pump 18, e.g., for suctioning through the outflow cannula tubing 28, the pump 10 drives up or activates the motor 46 to allow for suctioning through the outflow device tubing 32.

In response to determining 112 that the current exceeds the current threshold, the device activation indicator 74 is activated 118, such as by the circuit 70 providing an instruction to the device activation indicator 74.

The transmission 114 of the instruction signal and the activation 118 of the indicator 74 can occur in any order.

If the determination 112 that the current exceeds the current threshold is a first such determination in the method 100, and if the detector 56 is configured to configured to automatically set the current threshold, the detector 56 can automatically set the current threshold as discussed above since the detector 56 will now have enough data to calculate the current threshold, e.g., has data indicating the active current and data indicating the inactive current.

With the surgical device 34 in the active or actuated state such that suction being provided through the outflow device tubing 32, the detector 56 continues the process as described above, starting with the sensor 66 sensing 104 the AC power received at the detector 56 via the AC input power connector 64. Eventually, the current will be determined 112 to not exceed the current threshold, or the AC power 62 will stop being fed 102 to the detector 56 and thereby effectively stop the method 100 since the sensor 66 will not have any electrical power to sense.

If the comparison 110 determines 112 that the current does not exceed the current threshold, the surgical device 34 is presumed to be in the inactive or standby state in which the surgical device 34 is not operational, e.g., is not providing suction. The detector 56, e.g., the circuit 70 thereof, determines 120 whether an instruction signal was previously transmitted 114 to the pump 10. If so, the surgical device 34 is presumed to be in the active or actuated state. The detector 56 thus transmits 122 a second instruction signal to the pump 10 via the pump connector 76. In response to receiving the second instruction signal, the pump 10 activates 124 the pinch valve 54 so that suction ceases being provided through the outflow device tubing 32. The process then repeats starting with the sensor 66 sensing 104 the AC power received at the detector 56. Eventually, either the current will be determined 112 to exceed the current threshold with the method 100 continuing as discussed above, or the AC power 62 will stop being fed 102 to the detector 56 and thereby effectively stop the method 100 since the sensor 66 will not have any electrical power to sense.

If it is determined 120 that an instruction signal was not previously transmitted 114 to the pump 10, the surgical device 34 is presumed to be in the inactive state. The process then repeats starting with the sensor 66 sensing 104 the AC power received at the detector 56. Eventually, either the current will be determined 112 to exceed the current threshold with the method 100 continuing as discussed above, or the AC power 62 will stop being fed 102 to the detector 56 and thereby effectively stop the method 100 since the sensor 66 will not have any electrical power to sense.

The devices, systems, and methods described herein are not limited to use with surgical pumps as surgical source equipment. The devices, systems, and methods described herein can also be used, for example, with a generator as surgical source equipment. The generator can be an element of a radiofrequency (RF) system in which an RF surgical device (RF electrode) delivers RF energy to tissue, with the surgical device receiving the RF energy from the generator. One example of an RF system is the VAPR VUE® Radiofrequency Electrode System available from DePuy Mitek of Raynham, Mass. that includes the VAPR VUE® Generator and various VAPR VUE® Electrodes.

In general, in an RF system, a generator is operatively coupled to AC power and is operatively coupled to an RF surgical device configured to deliver RF energy to tissue using one or more electrodes. The AC power received by the generator is delivered to the RF surgical device to power the RF surgical device. Different amounts of power may be provided to the RF surgical device based on a current mode of operation of the RF surgical device. Thus, detecting an amount of AC power being provided to the RF surgical device using a detector can inform the generator which mode of operation the RF surgical device is currently in and thus how much power should be provided to the RF surgical device from the generator to achieve the desired effect of the RF surgical device's mode of operation. Examples of modes of operation of the RF surgical device include an ablation or vaporization mode in which high frequency power is delivered from the generator to the RF surgical device to cause tissue ablation, a coagulation mode of operation in which high frequency power is delivered from the generator to the RF surgical device to cause tissue coagulation, a modified ablation mode of operation in which high frequency power is delivered from the generator to the RF surgical device in short-duration pulses to cause more rapid tissue ablation than in the ablation mode of operation, a blended vaporization mode of operation that is similar to the ablation mode but also provides hemostasis, and a coagulation mode of operation with temperature control that is similar to the coagulation mode of operation except that temperature at the RF surgical device's tip is monitored and the power level adjusted to maintain the tip at a set temperature.

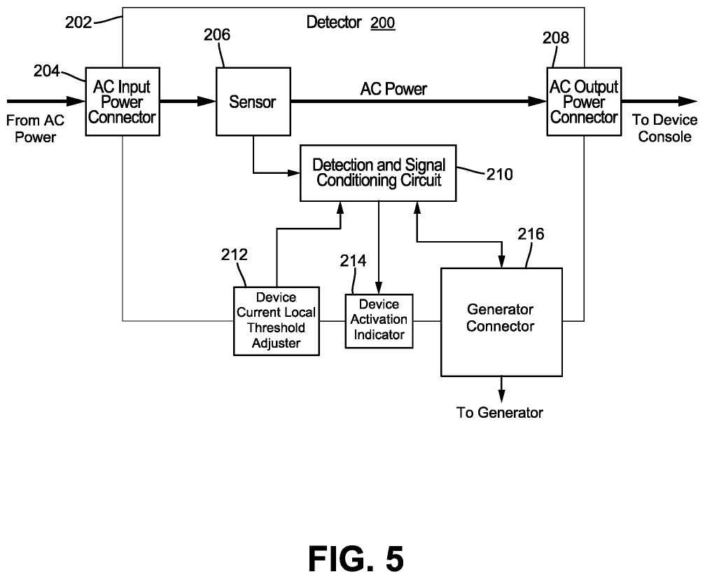

FIG. 5 illustrates one embodiment of a detector 200 configured to be used in an RF system. The detector 200 is generally configured and used similar to the detector 56 of FIGS. 1 and 2 that is discussed above and includes similarly-named and configured components as the detector 56, so the components of the detector 200 are identified below but their configuration and use is not again provided. The detector 200 is configured to be operatively connected to a generator, to an RF surgical device via a device console, and to AC power, e.g., an AC power source such as a wall outlet or other source. The detector 200 includes a housing 202, an AC input power connector 204, a sensor 206, an AC output power connector 208, a detection and signal conditioning circuit 210, a device current local threshold adjuster 212, a device actuation indicator 214, and a generator connector 216. The variations of the detector 56 and the pump 10 described above similarly apply to the detector 200 and the generator, e.g., the device current local threshold adjuster 212 may be omitted, the generator can include a device current remote threshold adjuster, the detector 200 can be configured to automatically set a current threshold, a current threshold can be adjustable or can be non-adjustable, etc.

The devices, systems, and methods described herein can be used with an RF shaver configured to provide suction, similar to the surgical device 34 discussed above, and to deliver RF energy to tissue using one or more electrodes similar to the RF surgical device discussed above. In such embodiments, a detector can operate similar to the detectors 56, 200. Various embodiments of RF shavers are further described, for example, in U.S. Pat. Pub. No. 2016/0058501 entitled “Cavitating Ultrasonic Surgical Aspirator With RF Electrodes” published Mar. 3, 2016, which are hereby incorporated by reference in its entirety.

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

What is claimed is:

1. A surgical system, comprising:
a detector configured to be operatively connected to each of an AC power source, a surgical pump, and a surgical device, the surgical device being configured to be releasably coupled to the surgical pump; wherein:
the detector includes a housing external to the pump and to the surgical device; and
the detector includes a sensor and a circuit housed in the housing;
the sensor is configured to sense electrical power received by the detector from the AC power source; and
the circuit is configured to determine if the sensed electrical power exceeds a threshold and, if the sensed electrical power is determined to exceed the threshold, to transmit a signal to the surgical pump that causes the surgical pump to provide suction pressure to the surgical device thereby allowing the surgical device to suction fluid from a surgical site.

2. The system of claim 1, wherein the sensed electrical power exceeding the threshold is indicative of the surgical device being actuated to suction fluid.

3. The system of claim 1, wherein the sensed electrical power exceeding the threshold is indicative of the surgical device being in an active state in which the surgical device is operational to provide aspiration; and
the sensed electrical power not exceeding the threshold is indicative of the surgical device being in an inactive state in which the surgical device is not operational to provide aspiration.

4. The system of claim 1, wherein the threshold is pre-programmed and stored at the detector.

5. The system of claim 1, wherein at least one of the detector and the surgical pump includes an adjuster configured to allow a user to set the threshold.

6. The system of claim 1, wherein the circuit is configured to dynamically set the threshold based on the sensed electrical power.

7. The system of claim 1, wherein the sensor is configured to repeatedly sense electrical power received over time by the detector from the AC power source; and
if the sensed electrical power is determined to not exceed the threshold, the circuit is configured to determine if a next sensed electrical power exceeds the threshold.

8. The system of claim 1, wherein the surgical pump includes an arthroscopic pump, and the surgical site is at a joint of a patient.

9. A surgical system, comprising:
a surgical device configured to be operatively connected to surgical source equipment configured to provide operational functionality to the surgical device operatively connected thereto, the operational functionality allowing the surgical device to perform a function at a surgical site;
a housing that is external to the surgical device and to the surgical source equipment and that is configured to be operatively coupled to the surgical device, to the surgical source equipment, and to an AC power source;
a sensor housed in the housing and configured to sense electrical power received at the housing from the AC power source; and
a circuit housed in the housing and configured to determine if the sensed electrical power exceeds a threshold and, if the sensed electrical power is determined to exceed the threshold, to transmit a signal to the surgical source equipment that causes the surgical source equipment to provide the operational functionality to the surgical device and thereby allowing the suction surgical to perform the function at the surgical site.

10. The system of claim 9, wherein the sensed electrical power exceeding the threshold is indicative of the surgical device being in an active state in which the surgical device is operational to perform the function; and
the sensed electrical power not exceeding the threshold is indicative of the surgical device being in an inactive state in which the surgical device is not operational to perform the function.

11. The system of claim 9, wherein the threshold is pre-programmed.

12. The system of claim 9, further comprising an adjuster configured to allow a user to set the threshold.

13. The system of claim 9, wherein the circuit is configured to dynamically set the threshold based on the sensed electrical power.

14. The system of claim 9, wherein the sensor is configured to repeatedly sense electrical power received over time at the housing from the AC power source; and
if the sensed electrical power is determined to not exceed the threshold, the circuit is configured to determine if a next sensed electrical power exceeds the threshold.

15. The system of claim 9, wherein t the surgical source equipment includes a surgical pump, the operational functionality includes a suction force, and the function includes suctioning fluid from the surgical site.

16. The system of claim 9, wherein the surgical device includes an electrode, the surgical source equipment includes a generator, the operational functionality includes RF energy, and the function includes delivering RF energy to tissue at the surgical site via the electrode.

17. A surgical method, comprising:
sensing, using a sensor housed in a housing of a detector, electrical power received at the detector from an AC power source, the detector being operatively coupled to a surgical pump and being external to the surgical pump;
determining, using a circuit housed in the housing of the detector, if the sensed electrical power exceeds a threshold; and
if the sensed electrical power is determined to exceed the threshold, transmitting a signal from the detector to the surgical pump that causes the surgical pump to provide suction pressure to a surgical device releasably coupled to the surgical pump such that the surgical device suctions fluid from a surgical site.

18. The method of claim 17, wherein the sensed electrical power exceeding the threshold is indicative of the surgical device being in an active state in which the surgical device is operational to provide aspiration; and
the sensed electrical power not exceeding the threshold is indicative of the surgical device being in an inactive state in which the surgical device is not operational to provide aspiration.

19. The method of claim 17, further comprising repeatedly sensing, using the sensor, electrical power received over time by the detector from the AC power source; and
if the sensed electrical power is determined to not exceed the threshold, determining, using the circuit, if a next sensed electrical power exceeds the threshold.

20. The method of claim 17, wherein the surgical pump includes an arthroscopic pump, and the surgical site is at a joint of a patient.
